(19) United States
(12) Patent Application Publication
(10) Pub. No.: US 2024/0416087 A1
(43) Pub. Date: Dec. 19, 2024

(54) NASAL INSERTS FOR TREATING EPISTAXIS

(71) Applicant: Creo Health LLC, Toledo, OH (US)

(72) Inventors: Robert Joseph Fisher, Lima, OH (US); Paul Martin Ferreira Leonard, Toledo, OH (US)

(73) Assignee: Creo Health LLC, Toledo, OH (US)

(21) Appl. No.: 18/642,025

(22) Filed: Apr. 22, 2024

Related U.S. Application Data
(60) Provisional application No. 63/460,931, filed on Apr. 21, 2023.

(51) Int. Cl. A61M 25/10; A61B 17/00; A61B 17/12; A61F 13/45; A61M 31/00

(52) U.S. Cl. CPC A61M 25/10; A61B 17/1204; A61B 17/12104; A61B 17/12136; A61F 13/45; A61M 31/00; A61B 2017/00893; A61B 2017/12004; A61M 2025/105; A61M 2025/1081; A61M 2210/0618

(57) ABSTRACT

A nasal insert system for treating epistaxis is disclosed. The nasal insert system includes a selectively expanding interior bladder, and a permeable exterior bladder formed of a first expandable member and a second expandable member, covered by a woven gauze sleeve. In one embodiment, the first and second expandable members comprise a plurality of pores in a top and front position corresponding to “little's area” of the nasal cavity.

### CROSS REFERENCE TO RELATED APPLICATIONS

This application claims the benefit of Provisional Application No. 63/460,931filed on Apr. 21, 2023 which is hereby incorporated herein by reference.

### TECHNICAL FIELD

This disclosure relates to nasal inserts, and more particularly to nasal inserts for treating epistaxis.

### BACKGROUND OF THE INVENTION

The statements in this section merely provide background information related to the present disclosure and may not constitute prior art.

Severe nosebleeds, called epistaxis, are generally difficult to treat, requiring treatment over a number of days. While epistaxis is usually cured by minimal intervention, it can become a serious medical issue if hemostasis is not achieved relatively quickly. Epistaxis is generally treated via various types of nasal packing including, e.g., cotton gauze, sponge, or cellulose, or some expandable material. Nasal packing treatment for epistaxis can be painful and traumatic to the nasal mucosa, and may necessitate hospitalization for several days.

Various methods and devices are known for attempting to stop or mitigate epistaxis via drug application. These known methods can include drug application to an exterior surface of a nasal packing material.

Other known devices use inflating devices to selectively expand within a nasal cavity. These devices generally consist of a balloon configured to press against the source of bleeding and assists the clotting of the blood in order to create hemostasis. It is known to place pro-clotting agents to the exterior of the balloon before inserting into the nasal cavity.

Known devices are disadvantaged in that they do not allow for selective application of medicine to particular surfaces of a selectively expanding member, and do not allow focused treatment to “little's area” of the nasal cavity. Known devices having balloons are also undesirably shaped, which can lead to undesirable application of medicine to nasal surfaces.

Hence, a need exists for a nasal insert for treating epistaxis that allows for targeted application of medicine to particular surfaces of a selectively expanding member.

### SUMMARY

A nasal insert system for treating epistaxis is disclosed. The system includes an expanding interior bladder configured to selectively receive ambient air, a permeable, expanding member configured to house the expanding interior bladder, wherein the permeable, expanding member is configured for selective communication of a medical fluid, and a tubular-shaped gauze sleeve fitted over the permeable, expanding member.

In one embodiment, a nasal insert includes a selectively expanding bladder, a first expandable member coupled to a first side of the bladder, and a second expandable member coupled to a second side of the bladder. In one embodiment, the first and second expandable members comprise a plurality of pores in a top and front position corresponding to “little's area” of the nasal cavity.

This summary is provided merely to introduce certain concepts and not to identify key or essential features of the claimed subject matter.

### BRIEF DESCRIPTION OF THE FIGURES

One or more embodiments will now be described, by way of example, with reference to the accompanying drawings, in which:

FIGS. 1A and 1B schematically show exemplary nasal insert systems, in accordance with the present disclosure;

FIG. 2A shows an exemplary medicine injection apparatus, in accordance with the present disclosure;

FIG. 2B shows an exemplary air injection apparatus, in accordance with the present disclosure;

FIG. 2C shows an exemplary nasal insert, in accordance with the present disclosure;

FIG. 3A is a cross-sectional view along line C-C of FIG. 1A of an embodiment of the nasal insert, in accordance with the present disclosure;

FIG. 3B is a cross-sectional view along line D-D of FIG. 1B of an embodiment of the nasal insert, in accordance with the present disclosure;

FIGS. 4A and 4B show another nasal insert embodiment, in accordance with the present disclosure;

FIG. 5 shows a cross-sectional view along line A-A of FIG. 4B, in accordance with the present disclosure;

FIG. 6 shows a cross-sectional view along line B-B of FIG. 4B, in accordance with the present disclosure;

FIGS. 7A and 7B show perspective views of embodiments of the nasal insert system, in accordance with the present disclosure; and FIG. 8 shows another embodiment of the nasal insert.

### DETAILED DESCRIPTION

Reference throughout this specification to “one embodiment,” “an embodiment,” or similar language means that a particular feature, structure, or characteristic described in connection with the embodiment is included in at least one embodiment of the subject matter of the present disclosure. Appearances of the phrases “in one embodiment,” “in an embodiment,” and similar language throughout this specification may, but do not necessarily, all refer to the same embodiment.

Various embodiments of the present invention will be described in detail with reference to the drawings, where like reference numerals represent like parts and assemblies throughout the several views. Reference to various embodiments does not limit the scope of the invention, which is limited only by the scope of the claims attached hereto. Additionally, any examples set forth in this specification are not intended to be limiting and merely set forth some of the many possible embodiments for the claimed invention.

As used in the description herein and throughout the claims, the following terms take the meanings explicitly associated herein, unless the context clearly dictates otherwise: the meaning of “in” includes “in” and “on.” The term “based upon” is not exclusive and allows for being based on additional factors not described, unless the context clearly dictates otherwise. Additionally, in the subject description, the word “exemplary” is used to mean serving as an example, instance or illustration. Any embodiment or design described herein as “exemplary” is not necessarily to be construed as preferred or advantageous over other embodiments or designs. Rather, use of the word exemplary is intended to present concepts in a concrete manner.

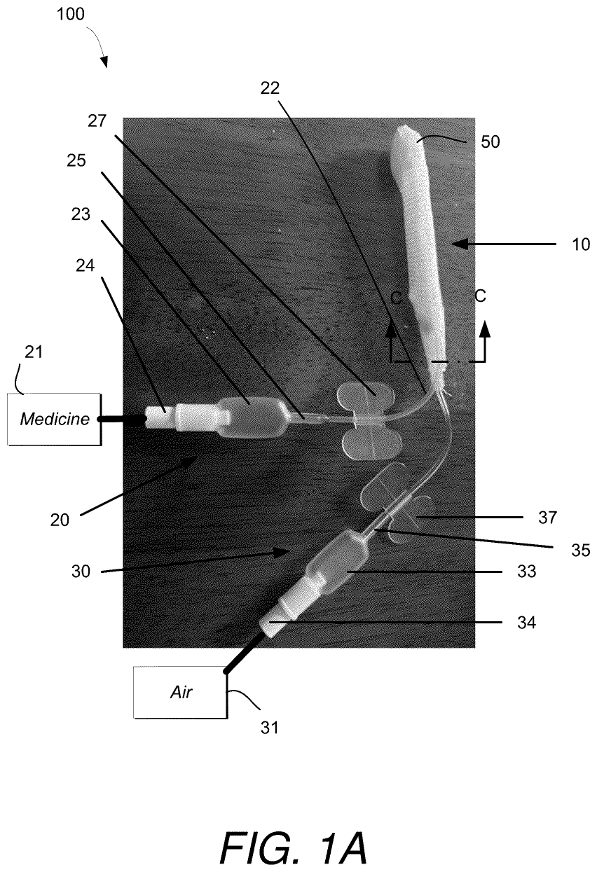

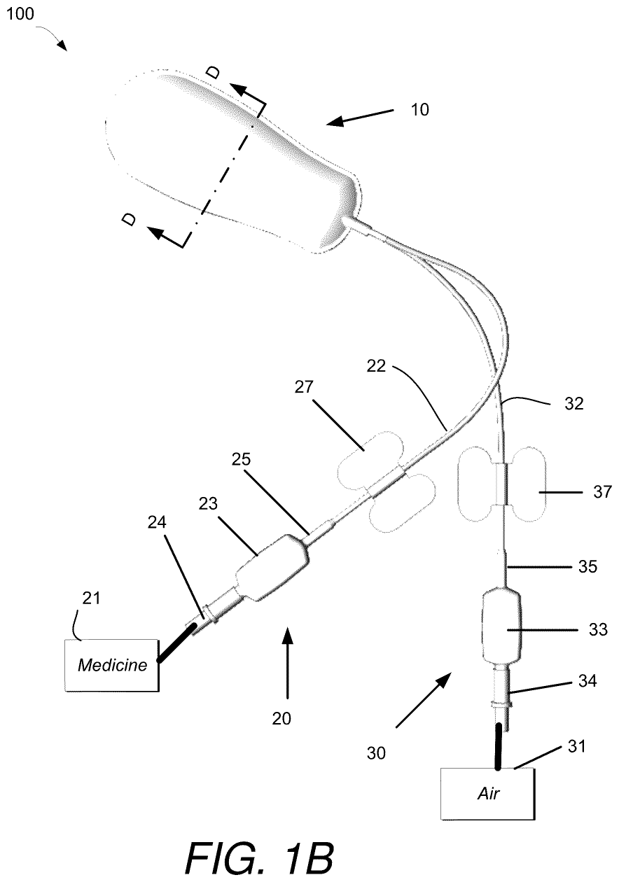

Referring now to the drawings, wherein the depictions are for the purpose of illustrating certain exemplary embodiments only and not for the purpose of limiting the same, FIGS. 1A and 1B schematically show exemplary nasal insert systems 100 for treating epistaxis that may help implement the methodologies of the present disclosure. The system 100 includes a nasal insert 10, a medicine injection apparatus 20, and an air injection apparatus 30. In various embodiments, the nasal insert 10 is not connected to one or both of the medicine injection apparatus 20 and the air injection apparatus 30. Some components of the system 100 are shown in FIGS. 1A and 1B as single elements. Such illustration is for ease of description and it should be recognized that the system 100 may include multiple additional devices and/or components. FIG. 1A shows an embodiment of the system 100 having a gauze sleeve 50 over a deflated balloon, while FIG. 1B shows the nasal insert 10 in an inflated state without a gauze sleeve.

In various embodiments, the medicine injection apparatus 20 includes a first port 24 that is configured for selective coupling to a medical delivery device 21. In various embodiments, the first port 24 may include a check valve, configured for one-way fluid communication of the medicine into the nasal insert 10. The first port 24 may be fluidly coupled to a flashback chamber 23 that is preferably transparent for viewing medicine communicating therethrough. The flashback chamber 23 may be connected to a bushing 25 or reducer conduit. In one embodiment, the medicine injection apparatus 20 includes stabilizing wings 27 configured to provide a surface area for taping down the apparatus 20, as desired. In one embodiment, the medicine injection apparatus 20 then includes the conduit 22 that may be coupled to the nasal insert.

In one embodiment, the conduit 22 is coupled to a first balloon 29 within the nasal insert 10. In one embodiment, the conduit 22 is coupled to a first balloon within the nasal insert 10 via a port.

In various embodiments, the air injection apparatus 30 includes a second port 34 that is configured for selective coupling to an air delivery device 31. In various embodiments, the second port 34 may include a two-way valve configured to permit selective air motion in both directions into the nasal insert 10. The second port 34 may be fluidly coupled to a flashback chamber 33, that may be configured to modulate air intake. The flashback chamber 33 may be connected to a bushing 35 or reducer conduit. In one embodiment, the air injection apparatus 30 includes stabilizing wings 37 configured to provide a surface area for taping down the apparatus 30, as desired. In one embodiment, the air injection apparatus 30 then includes the conduit 32 that may be coupled to the nasal insert.

In one embodiment, the conduit 32 is coupled to an expandable member, i.e., a second balloon 39 within the nasal insert 10. In one embodiment, the conduit 32 is coupled to a second balloon within the nasal insert 10 via a port.

The medicine injection apparatus 20 is preferably configured to selectively inject medicine into the nasal insert 10. The medicine injection apparatus 20 can include a delivery device, such as a syringe, for selectively communicating medicine or fluids to the nasal insert 10. In one embodiment, the medicine injection apparatus 20 is a syringe with medicine.

The air injection apparatus 30 is preferably configured to selectively inject air into the nasal insert 10. The air injection apparatus 30 can include a delivery device, such as a syringe, for selectively communicating air into the nasal insert 10. It is contemplated that the air injection apparatus 30 may be used to selectively communicate another fluid or gas consistent with the teachings herein. The air is communicated to the nasal insert 10 for selectively inflating a bladder for a tamponade effect with a patient's nasal cavity, as described herein below. In one embodiment, the air injection apparatus 30 is a syringe.

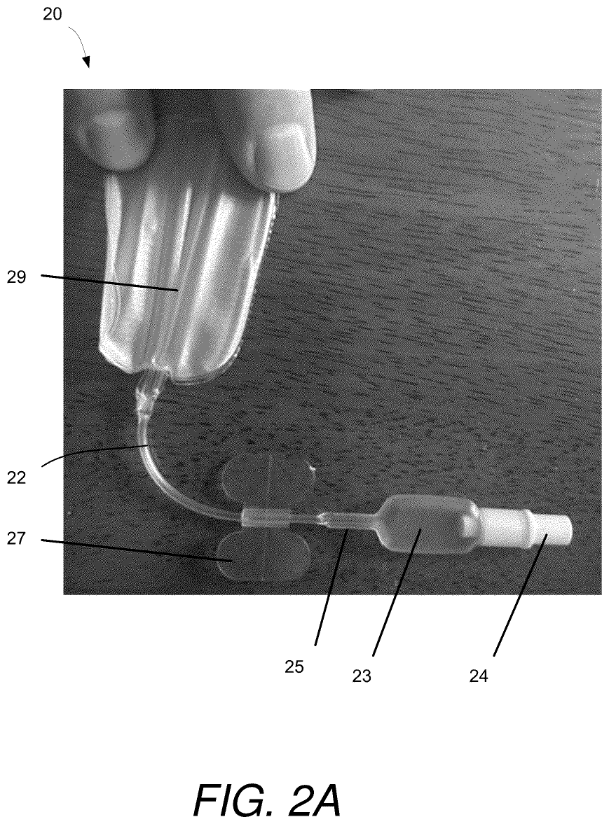

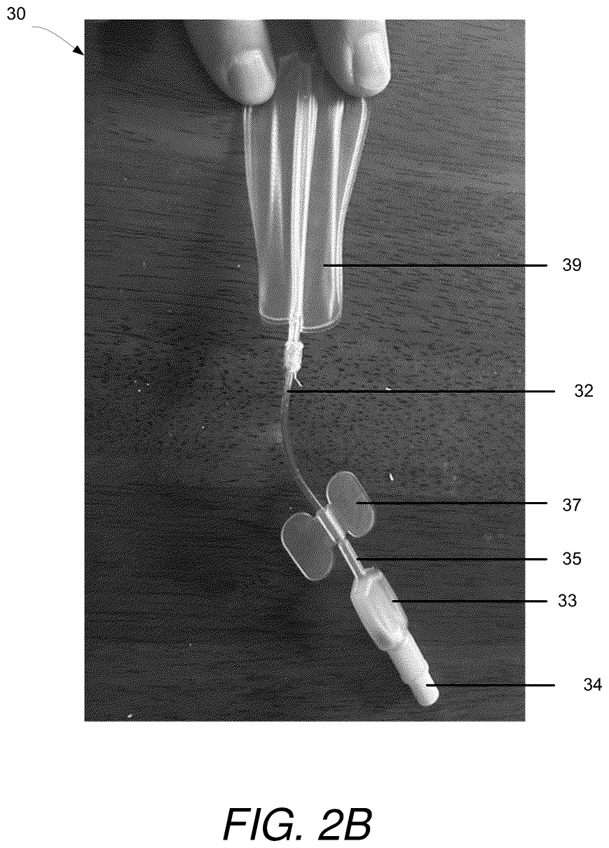

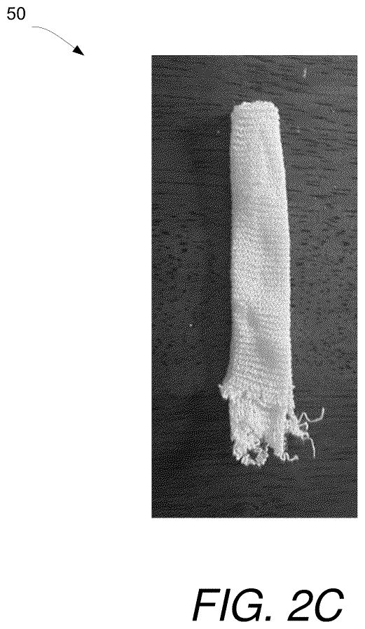

FIG. 2A shows an exemplary medicine injection apparatus 20 and a first balloon 29. As FIG. 2A shows, the conduit 22 is fluidly connected to the first balloon 29. In one embodiment, the conduit 22 may be inserted into an opening of the balloon 29. In one embodiment, the conduit attaches to a port connected to the balloon 29. Silicon-based epoxy or other sealant may be used to create a non-permeable seal.

FIG. 2B shows an exemplary air injection apparatus 30 and a second balloon 39. As FIG. 2B shows, the conduit 32 is fluidly connected to the second balloon 39. In one embodiment, the conduit 32 may be inserted into an opening of the second balloon 39. In one embodiment, the conduit attaches to a port connected to the second balloon 39. Silicon-based epoxy or other sealant may be used to create a non-permeable seal. As FIG. 2B shows, the second balloon 39 is permeable, configured for selective transmission of the medicine from the inside of the second balloon 39 to the exterior when an internal pressure is exerted thereon. Such a pressure may be applied, for example, by the first balloon 29 being inside of the second balloon 39 and expanding against the interior walls of the second balloon 39, pushing the medicine through the walls of the second balloon 39.

FIG. 2C shows an exemplary gauze sleeve 50. As FIG. 2C shows, the gauze sleeve 50 is preferably formed of a thin, fabric with a loose weave, thereby creating small holes from which medicine may pass through. In various embodiments, the gauze sleeve 50 may be coated with an antimicrobial compound and/or a pro-clotting compound such as carboxycellulose or the like.

In one embodiment, the nasal insert 10 includes a gauze sleeve 50, the first balloon 29 and the second balloon 39. In one embodiment, the first balloon 29 is a bladder. In one embodiment, the second balloon 39 is expandable. The first balloon 29 is situated within the second balloon 39. In one embodiment, the first balloon 29 is coupled to the and the second balloon 39, which may be accomplished using an adhesive.

The first balloon 29 can be formed of an elastomeric material which could be tubular-shaped or cylindrical-shaped. Various elastomer materials may be used to form the first balloon 29 according to preferential pressure-volume characteristics within the dimensions of the first balloon 29 for imparting a tamponade effect to surfaces within a nasal cavity. The elastomer materials is preferably non-permeable, with the exception of the port 34. In one embodiment, the first balloon 29 is formed of a rubber polymer. In one embodiment, the first balloon 29 is formed of a silicon-based polymer and/or includes a silicon-based liner.

The second balloon 39 can be formed of any number of expandable materials. The second balloon 39 can be formed of material configured to absorb a medicine, e.g., thrombostatic medicine, and then diffuse the medicine outwardly when the material undergoes pressure. In one embodiment, the second balloon 39 is formed of a sponge material. In one embodiment, the second balloon 39 is formed of a cellulose material. In one embodiment, the second balloon 39 is formed of a mesh material. In one embodiment, the second balloon 39 is formed of a foam material. In one embodiment, the second balloon 39 is formed of an anti-bacterial material. In one embodiment, the second balloon 39 is formed of an anti-bacterial polyurethane foam.

The nasal insert 10 is shown in FIGS. 1-2C in an unexpanded state.

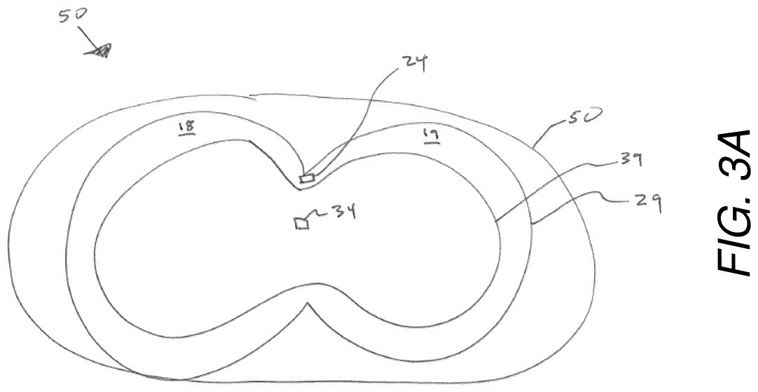

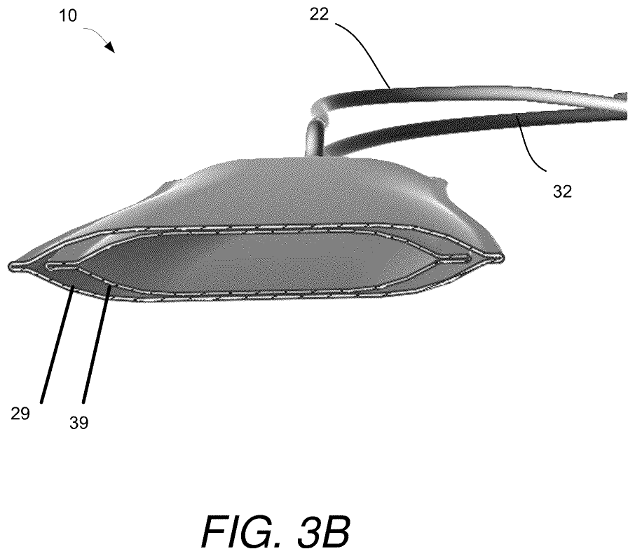

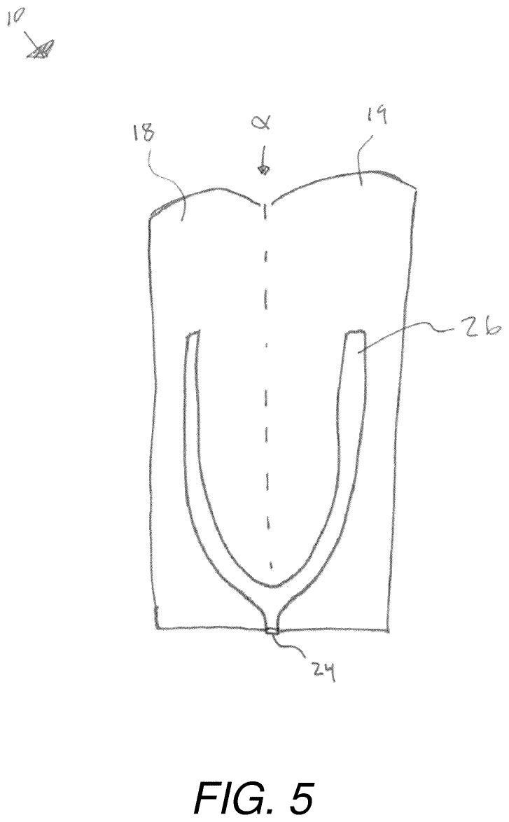

FIG. 3A is a cross-sectional view along line C-C of FIG. 1A. As FIG. 3A shows, the first balloon 29 may be formed of a left-side expandable member 18 and a right-side expandable member 19. The left and right expandable members 18 and 19 may be integral, i.e., a single, congruous expandable member. In one embodiment, the left and right expandable members 18 and 19 are in fluid communication, that is medicine may move freely within and between the members 18 and 19. In one embodiment, the left and right expandable members 18 and 19 may be coupled to the first balloon 29 along a pinch line, e.g., a, (also shown in FIG. 5) that traverses from a front portion of the nasal insert 10 to a rear portion.

The second balloon 39 may also be formed of a left and right expandable members. In one embodiment, the left and right expandable members of the second balloon 39 are sized and shaped to abut the interior surface walls of the first balloon 29 when in an expanded state.

FIG. 3B is a cross-sectional view of the nasal insert 10 without the gauze sleeve 50 along line D-D of FIG. 1B. As FIG. 3B shows, the first and second balloons 29 and 39, respectively, may each have a substantially oval cross-sectional shape.

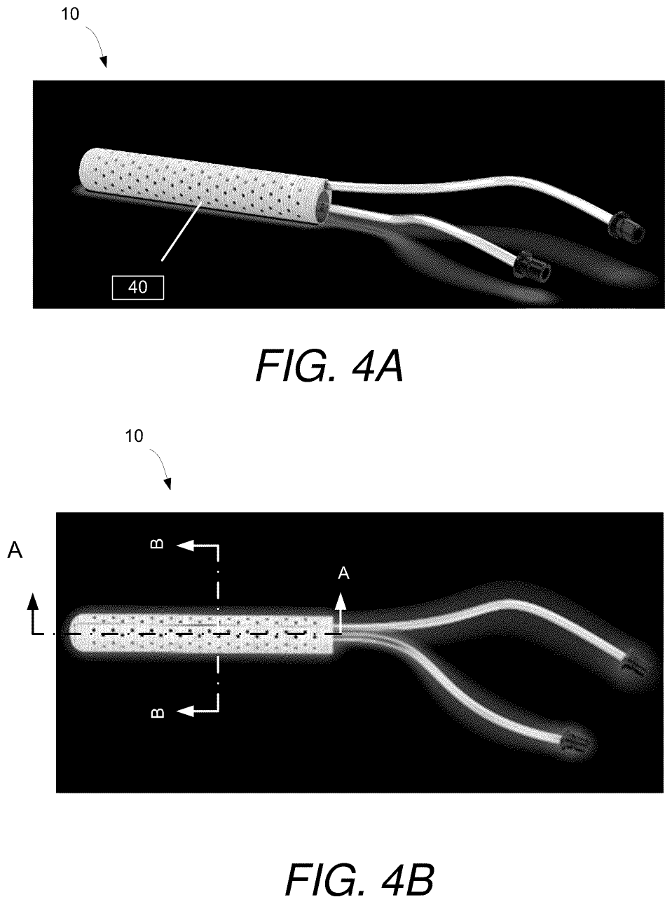

FIGS. 4A and 4B show a view of the nasal insert 10 without the gauze sleeve 50, the left-side being a substantially mirror image thereof. As FIGS. 4A and 4B show, the left and right expandable members 18 and 19 of the first balloon 29 can include a plurality of pores 40. The pores are configured to aid medicine diffusion from within the respective expandable members 18 and 19. In one embodiment, the pores 40 traverse the first balloon 29 from the surface to a conduit within the first balloon 29 as described herein below. In one embodiment, the pores 40 are formed by removing material from the expandable portion at desirable areas. In one embodiment, the pores 40 are located on the left and right expandable members 18 and 19 to correspond to “little's area” of the nasal cavity. Consistent with placement proximate to “little's area” when inserted into the nasal cavity, in one embodiment, the pores are located in a front half of the nasal insert 10. In one embodiment, the pores 40 are located in a top, front portion of the left and right expandable members 18 and 19. In one embodiment, the pores 40 are evenly spaced perforations to allow the selected medical fluid to extravasate and saturate the gauze 50. In one embodiment, the pores 40 are directed to a top surface of the first balloon 29.

FIG. 5 shows a cross-sectional view along line A-A of FIG. 4B illustrating an embodiment of the nasal insert having an interior conduit 26. As FIG. 5 shows, the nasal insert 10 includes an interior conduit 26 configured to communicate injected medicine to both the left and right expandable members 18 and 19 of the second balloon 39. The interior conduit 26 can be permeable so that the medicine can diffuse throughout the left and right expandable members 18 and 19. In one embodiment, the interior conduit 26 has a substantially tubular shape and is formed of a polymer-based material. In one embodiment, the interior conduit 26 is connected to the conduit 22, via a port. In another embodiment, the interior conduit 26 is integral with the conduit 22. In one embodiment, the interior conduit 26 is a continuation of elongated conduit 22 within the first balloon 29

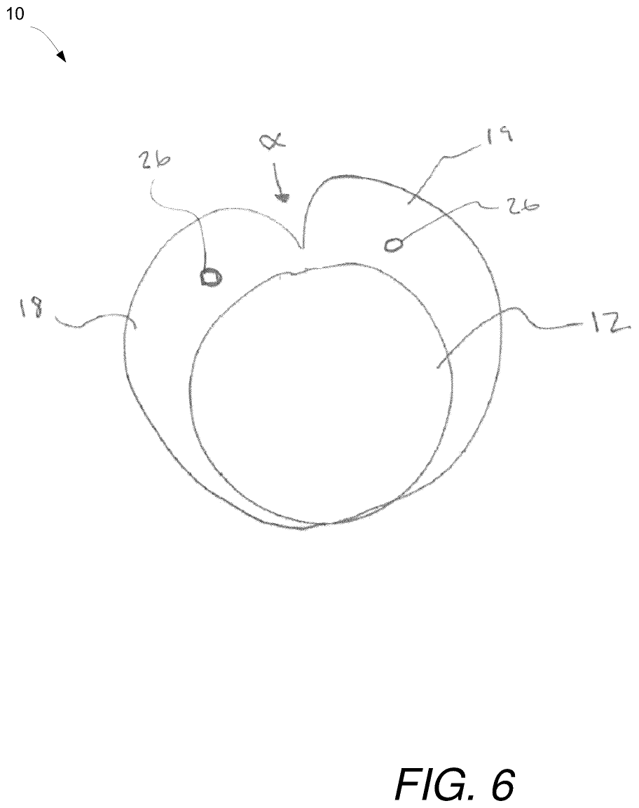

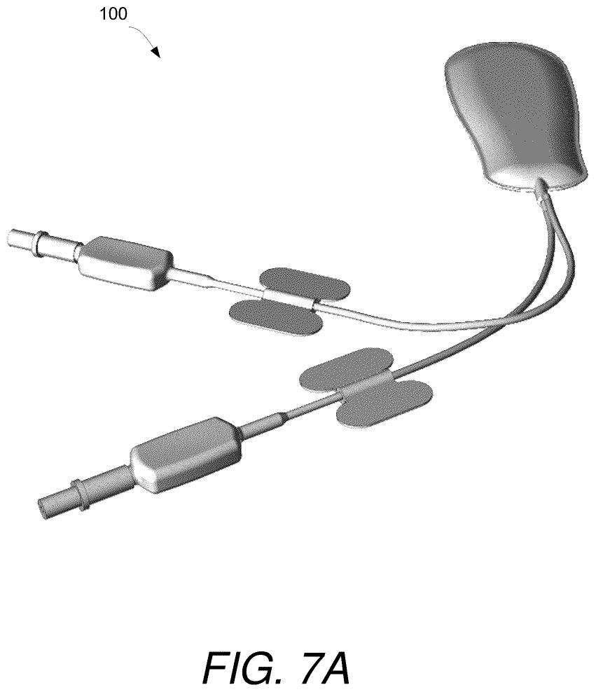

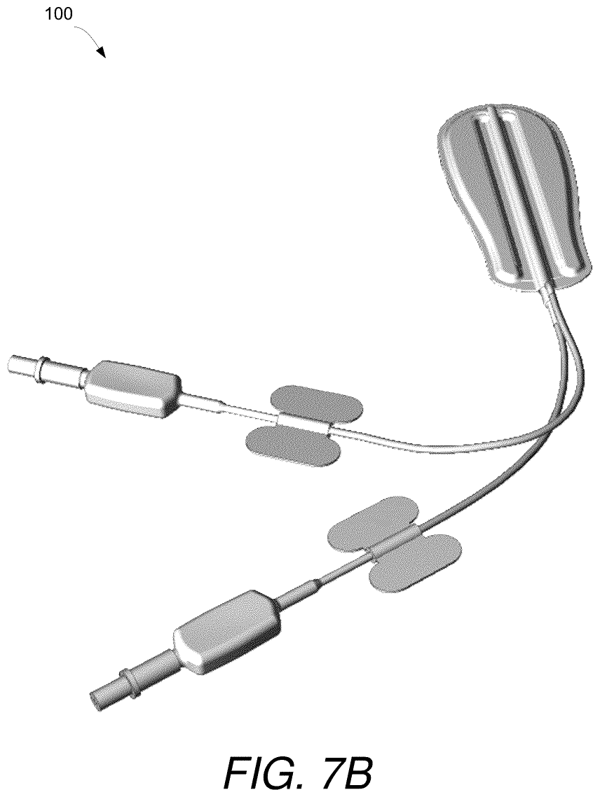

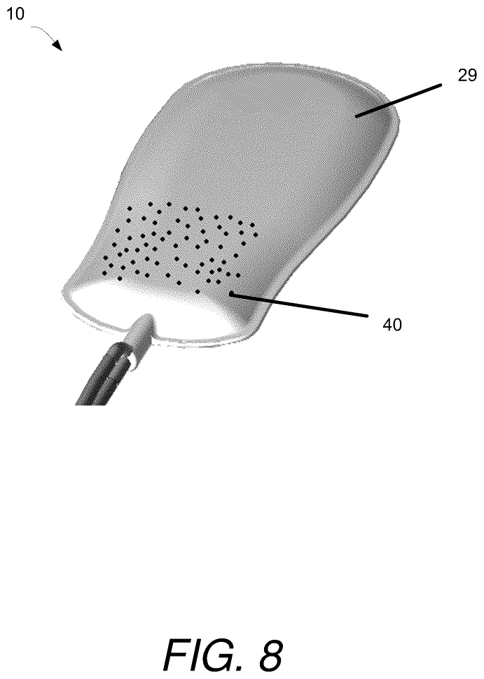

FIG. 6 shows a cross-sectional view along line B-B of FIG. 4B. As FIG. 6 shows, the interior conduit 26 is within the left and right expandable members 18 and 19 of the second balloon 39. In one embodiment, the second balloon 39 is formed of integral heart cross-sectional shaped portion. In one embodiment, the second balloon 39 is formed of integral figure-eight shaped cross-sectional shaped portion.

In use, the nasal insert 10 is inserted into a nasal cavity of a patient. Medicine to treat epistaxis may be injected into the left and right expandable members 18 and 19 via the medicine injection apparatus 20, the conduit 22, through the first port 24, and into the interior conduit 26. Once left and right expandable members 18 and 19 are saturated with medicine, e.g., thrombostatic medicine, the first balloon 29 may be pressurized via the air injection apparatus 30, conduit 32, and second port 34. Once expanded, the first balloon 29 provides a tamponade effect, pushing the medicine infused expandable members 18 and 19 against the nasal walls, inhibiting and then stopping bleeding.

FIG. 8 shows an embodiment of the nasal insert 10. For ease of illustration, the insert 10 is shown without the gauze sleeve 50. As FIG. 8 shows, the first balloon 29 may include a plurality of pores 40 in a top and front position corresponding to “little's area” of the nasal cavity. In one embodiment, the pores 40 are a plurality of localized pores associated with an area less than half of a top surface area of the permeable, first balloon 29. In one embodiment, the permeable, first balloon 29 includes a plurality of evenly spaced perforations, i.e., pores. The pores 40 are sized and shaped to allow a selected medical fluid to extravasate and saturate the gauze sleeve.

Additionally, examples in this specification where one element is “coupled” to another element can include direct and indirect coupling. Direct coupling can be defined as one element coupled to and in some contact with another element. Indirect coupling can be defined as coupling between two elements not in direct contact with each other, but having one or more additional elements between the coupled elements. Further, as used herein, securing one element to another element can include direct securing and indirect securing.

In the above description, certain terms may be used such as “left,” “right,” and the like. These terms are used, where applicable, to provide some clarity of description when dealing with relative relationships. But, these terms are not intended to imply absolute relationships, positions, and/or orientations. Nevertheless, it is still the same object. Further, the terms “including,” “comprising,” “having,” and variations thereof mean “including but not limited to” unless expressly specified otherwise. An enumerated listing of items does not imply that any or all of the items are mutually exclusive and/or mutually inclusive, unless expressly specified otherwise. The terms “a,” “an,” and “the” also refer to “one or more” unless expressly specified otherwise. Further, the term “plurality” can be defined as “at least two.”

While the foregoing disclosure discusses illustrative embodiments, it should be noted that various changes and modifications could be made herein without departing from the scope of the described embodiments as defined by the appended claims. Accordingly, the described embodiments are intended to embrace all such alterations, modifications and variations that fall within scope of the appended claims. Furthermore, although elements of the described embodiments may be described or claimed in the singular, the plural is contemplated unless limitation to the singular is explicitly stated. Additionally, all or a portion of any embodiment may be utilized with all or a portion of any other embodiments, unless stated otherwise.

1. A nasal insert system for treating epistaxis, the system comprising:
an expanding interior bladder configured to selectively receive ambient air;
a permeable, expanding member configured to house the expanding interior bladder, wherein the permeable, expanding member is configured for selective communication of a medical fluid; and
a tubular-shaped gauze sleeve fitted over the permeable, expanding member.

2. The nasal insert system of claim 1, wherein the permeable, expanding member is formed of an elongated first portion and an elongated second portion, wherein the elongated first portion and the elongated second portion form along a pinch line.

3. The nasal insert system of claim 2, wherein the elongated first portion and the elongated second portion each have a substantially tubular interior shape.

4. The nasal insert system of claim 3, wherein the elongated first portion and the elongated second portion are in fluid communication.

5. The nasal insert system of claim 4, wherein the permeable, expanding member has a heart-shaped cross-sectional shape.

6. The nasal insert system of claim 4, wherein the permeable, expanding member has a substantially figure-eight-shaped cross-sectional shape.

7. The nasal insert system of claim 4, wherein the permeable, expanding member has a substantially oval-shaped cross-sectional shape.

8. The nasal insert system of claim 4, wherein the permeable, expanding member has a substantially circular-shaped cross-sectional shape.

9. The nasal insert system of claim 2, wherein the permeable, expanding member comprises a plurality of pores in a top and front position corresponding to “little's area” of the nasal cavity.

10. The nasal insert system of claim 2, wherein the permeable, expanding member comprises a plurality of localized pores associated with an area less than half of a top surface area of the permeable, expanding member.

11. The nasal insert system of claim 2, wherein the permeable, expanding member comprises a plurality of pores comprising evenly spaced perforations configured to allow a selected medical fluid to extravasate and saturate the gauze sleeve.

12. The nasal insert system of claim 2, wherein the gauze sleeve comprises a fabric with a loose weave, having holes from which medicine may pass through.

13. A nasal insert system for treating epistaxis, the system comprising:
a medicine injection apparatus including:
a first port configured selective coupling to a medical delivery device, the first port includes a check valve, configured for one-way fluid communication of the medicine into a nasal insert,
a flashback chamber fluidly connected to the first port configured for viewing medicine communicating therethrough,
a conduit configured to communicate medicine into the nasal insert, and
stabilizing wings configured to provide a surface area for taping down the medicine injection apparatus;
an air injection apparatus including:
a second port configured for selective coupling to an air delivery device, the second port comprising a two-way valve configured to permit selective air motion in both directions into the nasal insert, and
a second pair of stabilizing wings configured to provide a surface area for taping down the air injection apparatus,
a second conduit coupled to the nasal insert; and
wherein the nasal insert comprises:
an expanding interior bladder configured to selectively receive ambient air;
a permeable, expanding member configured to house the expanding interior bladder,
wherein the permeable, expanding member is configured for selective communication of a medical fluid; and
a tubular-shaped gauze sleeve fitted over the permeable, expanding member.

14. The nasal insert system of claim 13, wherein the permeable, expanding member is formed of an elongated first portion and an elongated second portion, wherein the elongated first portion and the elongated second portion form along a pinch line.

15. The nasal insert system of claim 14, wherein the elongated first portion and the elongated second portion each have a substantially tubular interior shape.

16. The nasal insert system of claim 15, wherein the elongated first portion and the elongated second portion are in fluid communication.

17. The nasal insert system of claim 16, wherein the permeable, expanding member has a heart-shaped cross-sectional shape.

18. The nasal insert system of claim 16, wherein the permeable, expanding member has a substantially figure-eight-shaped cross-sectional shape.

19. The nasal insert system of claim 16, wherein the permeable, expanding member has a substantially oval-shaped cross-sectional shape.

20. The nasal insert system of claim 16, wherein the permeable, expanding member has a substantially circular-shaped cross-sectional shape.

21. The nasal insert system of claim 14, wherein the permeable, expanding member comprises a plurality of pores in a top and front position corresponding to “little's area” of the nasal cavity.

22. The nasal insert system of claim 14, wherein the permeable, expanding member comprises a plurality of localized pores associated with an area less than half of a top surface area of the permeable, expanding member.

23. The nasal insert system of claim 14, wherein the permeable, expanding member comprises a plurality of pores comprising evenly spaced perforations configured to allow a selected medical fluid to extravasate and saturate the gauze sleeve.

24. The nasal insert system of claim 14, wherein the gauze sleeve comprises a fabric with a loose weave, having holes from which medicine may pass through.
